(19) United States
(12) Patent Application Publication
(10) Pub. No.: US 2025/0180501 A1
(43) Pub. Date: Jun. 5, 2025

(54) Intelligent Electronic Nose System

(71) Applicant: University of Notre Dame du Lac, South Bend, IN (US)

(72) Inventors: Bingxin Yang, South Bend, IN (US); Nosang Myung, South Bend, IN (US); Faraj Al-badani, South Bend, IN (US); Thomas F. Stahovich, Riverside, CA (US)

(21) Appl. No.: 18/842,683

(22) PCT Filed: Mar. 3, 2023
(86) PCT No.: PCT/US2023/063689

§ 371 Date: Aug. 29, 2024

Related U.S. Application Data
(60) Provisional application No. 63/268,814, filed on Mar. 3, 2022.

(51) Int. Cl. G01N 27/14; G01N 27/26; G16C 20/70

(52) U.S. Cl. CPC G01N 27/14; G01N 27/26; G16C 20/70

(57) ABSTRACT

A detection device to detect analytes includes a sensor array and a controller. The sensor array includes a plurality of sensors, the plurality of sensors includes a sensing element, a heating element, and a lighting element. The controller is communicatively coupled to the sensor array, the heating element, and the lighting element, and configured to adjust at least one of the heating element or the lighting element based on a temperature profile of the heating element and an illumination profile of the lighting element.

### STATEMENT REGARDING FEDERALLY SPONSORED RESEARCH OR DEVELOPMENT

This invention was made with government support under grant N64267-19-C-0024 awarded by the Naval Sea Systems Command and grant NB18-21-27 awarded by US Air Force Research Laboratory (AFRL). The government has certain rights in the invention.

### TECHNICAL FIELD

The present description relates generally to detecting analytes using high performance sensing materials and more particularly to an intelligent electronic nose system.

### BACKGROUND

Military and civilian personnel are frequently required to perform their duties in complex and hazardous environments (e.g., an active warzone, post-disaster relief). However, it is usually infeasible for medical personnel to continuously assess the health status of personnel in such conditions. Likewise, it is often infeasible to dedicate time to collect samples of air or other materials from the hazardous environment for laboratory analysis. To determine if it is safe to enter and work, personnel must often conduct tests within the hazardous environment to detect various substances of interest (e.g., toxic industrial chemicals (TICs)). Therefore, it is desirable to have an automated device that can check for the presence of certain hazardous gaseous analytes or other materials without endangering personnel and without delaying operations due to lab processing.

### BRIEF DESCRIPTION OF THE DRAWINGS

FIG. 1 is a diagram of an example detection device for detecting various substances of interest in accordance with the various examples disclosed herein.

FIG. 2 is a block diagram illustrating an example system for training a machine learning model in accordance with the various examples disclosed herein.

FIG. 3 is a diagram of an example wearable/portable device that includes the detection device of FIG. 1 in accordance with the various examples disclosed herein.

FIG. 4 is a is a flow chart illustrating an example method of detecting various substances of interest in accordance with the various examples disclosed herein.

FIG. 5 is a flow chart illustrating an example method of classifying various substances of interest in accordance with the various examples disclosed herein.

### DETAILED DESCRIPTION

Because it is typically infeasible to use medical personnel to assess the health status of personnel in hazardous environmental conditions, an unattended monitoring tool is desirable. Furthermore, current laboratory-based methods used to detect substances of interest in these hazardous environments are frequently time consuming to operate. As such, a tool that can be used to quickly identify and detect substances of interest is advantageous.

The following disclosure of example methods and apparatus is not intended to limit the scope of the description to the precise form or forms detailed herein. Instead, the following disclosure is intended to be illustrative so that others may follow its teachings.

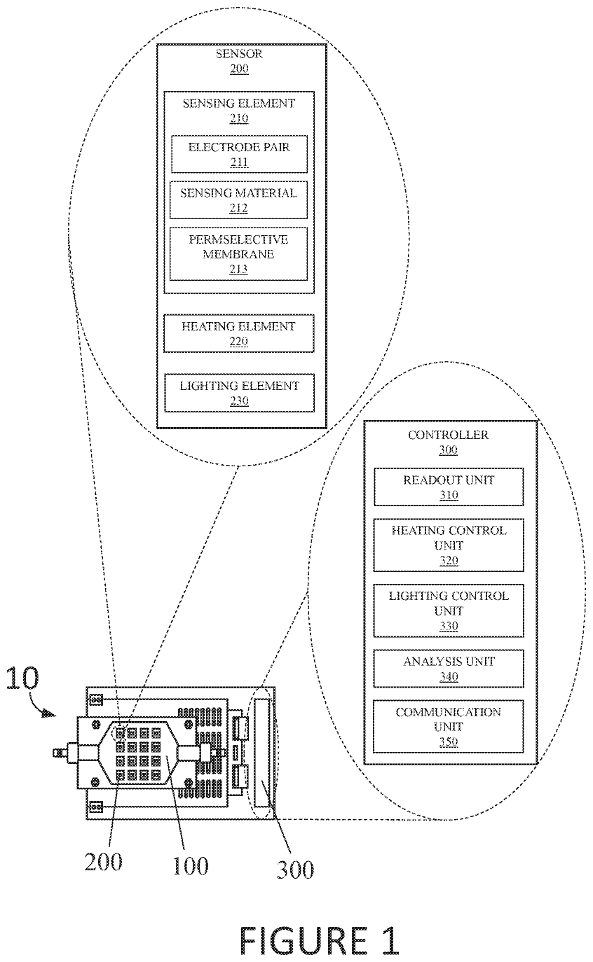

FIG. 1 is a diagram of an example detection device 10 for detecting gaseous target analytes. As shown in FIG. 1, the example detection device 10 includes a sensor array 100 containing one or more sensors 200 and a controller 300. As shown in FIG. 1, the sensor 200 of the detection device 10 comprises a sensing element 210, a heating element 220, and a lighting element 230. The sensing element 210 of the sensor 200 comprises an electrode pair 211, a sensing material 212, and a permselective membrane 213. The sensing material 212 electrically bridges the electrode pair 211.

The sensing material 212 is any suitable material for use in an electrical sensor (e.g., chemiresistor, chemicapacitor, impedimetric sensor) such that the sensing material 212 changes its electrical properties (e.g., resistance, capacitance, or impedance) in response to changes in the nearby chemical environment (e.g., direct chemical interaction between the sensing material 212 and a target analyte, or substance of interest, in the chemical environment). The sensing material 212 and the analyte may interact by physical or chemical adsorption and desorption, chemical reaction (e.g., catalytic oxidation or reduction), or molecular recognition (e.g., covalent bonding, hydrogen bonding, and Val der Waals interaction). Based on the changes caused by this molecular interaction, an output of the sensing material 212 can be used to evaluate the presence (or lack thereof) of a particular analyte in the air or other ambient atmosphere.

As used herein, analyte may refer to any substance whose chemical constituents are being identified and measured. The substance may be a chemical substance (e.g., ammonia, hydrogen sulfide, etc.), a gas, a vapor, a fume, an odor, or smell, for example. As such, while reference in this disclosure may be made to the analyte as a chemical or hazardous material, the disclosure should not be read as limited to such and should instead be read as applicable to any sensible substance contained in a gaseous environment. Furthermore, while reference is made primarily to a single analyte being sensed (e.g., “target analyte”), this disclosure should not be read as limited to the sensing of a single analyte and should instead be read as applicable to the sensing of one or more analytes at a time.

The sensing material 212 may include metal oxides (e.g., tin dioxide, chromium titanium oxide, gallium oxide, indium oxide, molybdenum oxide, tungsten oxide, or zinc oxide), transition metal dichalcogenides (TMDC), metals (e.g., gold, silver, platinum, palladium), metal organic frameworks (MOF), phyllosilicates (e.g., kaolinites), conductive polymers (e.g., polyaniline, polypyrrole), or carbonaceous nanostructures (e.g., single walled carbon nanotubes, graphene, graphene oxide). In some examples, the sensing material 212 may be an electrospun nanofiber. For example, the sensing material 212 may be a tungsten trioxide-based or tin oxide-based nanofiber, generated via electrospinning. The metal oxide-based nanofiber may be doped with a noble metal, such as silver, gold, palladium, platinum, ruthenium, strontium, or any other noble metal. In some examples, electrospinning is used to fabricate tungsten trioxide heterojunctions and other carbonaceous materials for use as a sensing material 212. In some examples, metal dopant is prepared from mixing a metal precursor (e.g., gold chloride) to a solvent (e.g., dimethylformamide). In some examples, the metal dopant is applied during the post treatment processing after electrospinning.

In some examples, the sensing material 212 is formed via electrospinning. In some examples, the electrospinning process includes a solution, a syringe for holding and dispensing the solution, and a collector for collecting the solution dispensed from the syringe. In some examples, a charged polymer-based precursor solution is ejected through a small orifice of the needle of the syringe under the effect of a high voltage electric field. The ejected solution may solidify or coalesce into a filamentous morphology. The polymer-based precursor solution in the syringe may be charged via the conductive metal needle, which may be electrically connected to a high voltage power supply. In some examples, as the precursor solution is slowly pushed out from the needle, the high-voltage electric field applied between the needle and the collector provides the electrostatic repulsive force that overcomes the surface tension of the droplet formed at the needle orifice to eject the charged precursor solution towards an electrically grounded collector. When the ejected solution travels to the grounded collector, the solution jet may solidify with the evaporation of the solvent such that solid nanofibers are deposited onto the collector. The electrospinning may be followed by post treatment processing, such as thermal treatment (e.g., calcination) and chemical modifications, to tune the composition, morphology, dimensions, crystallinity, and crystal structures of the sensing material 212.

In some examples, the sensing material 212 is a composite material comprising two or more materials combined together. For instance, in one example the sensing material comprises an electrospun metal oxide nanofiber coated with a noble metal dopant prepared from a solution containing both metal oxide (e.g., tin chloride) and noble metal (e.g., gold chloride) precursors.

The permselective membrane 213 comprises a membrane that allows only some substances (e.g., target analyte) to pass through the membrane and interact with the sensing material 212 while blocking other molecules from interacting with the sensing material 212. In this way, the permselective membrane is designed to at least partially control to which analyte(s) the sensing element 210 is sensitive. The permselective membrane may be an organic (e.g., polymer with ionic side groups (e.g., ion-exchange resins)) or a covalent organic framework (COF) or crystalline micro/mesoporous hybrid materials (e.g., metal organic framework (MOF)). In some examples, permselective membrane 213 is omitted.

Although reference is made throughout to a single sensing material 212 and/or a single permselective membrane 213, this disclosure should not be read as limited to inclusion of a single sensing material 212 and a single permselective membrane 213 but should instead be read as applicable to embodiments in which multiple sensing materials 212 and multiple permselective membranes 213 are included in the sensing element. Furthermore, the various sensors 200 comprising the sensor array 100 may each employ different sensing materials 212 and different permselective membranes 213.

The sensing performance of the sensing material 212 depends on its temperature. For example, the sensing material 212 may react more intensely (e.g., undergo a greater change in electrical properties) to a particular analyte at a higher temperature than at a lower temperature. Accordingly, each sensor 200 contains a heating element 220 which is used to adjust the sensing performance of the sensing material 212. The example heating element 220 is any suitable heating element configured to generate heat and to provide (e.g., direct, aim, guide, broadcast, etc.) that generated heat to the sensing material 212. For example, the heating element 220 may be a microheater made of platinum, gold, silver, nichrome, nickel, tungsten, titanium, aluminum, copper, graphene, carbon nanotubes, or other suitable material. In some examples, the heating element 220 is made of metal alloys such as titanium nitride, gallium nitride, gallium arsenide, Dilver P1 (an alloy of nickel, cobalt, and iron), polysilicon, or any other suitable metal alloy. Although reference is made to the heating element 220 as a single component, this disclosure should not be read as limited to the heating element 220 being a single heating element, such that this disclosure includes the heating element 220 being made of or including multiple heating elements (e.g., a heater with multiple coils, etc.).

The sensing performance of the sensing material 212 depends on the intensity and wavelengths of the light incident upon the sensing material 212. For example, the sensing material 212 may react more intensely (e.g., undergo a greater change in electrical properties) to a particular analyte when exposed to light with a shorter wavelength than when exposed to light with a longer wavelength. Accordingly, each sensor 200 contains a lighting element 230 which is used to adjust the sensing performance of the sensing material 212. The example lighting element 230 is any suitable lighting element configured to generate light with various wavelengths and intensity and to provide (e.g., direct, aim, guide, broadcast, shine, etc.) that generated light onto the sensing material 212. For example, the lighting element 230 may be one or more light-emitting diodes (LEDs) and/or diode lasers, although other suitable lighting elements may be utilized. Although reference is made to the lighting element 230 as a single component, this disclosure should not be read as limited to a single lighting element but should be read as including a lighting element having multiple lighting elements (e.g., an array with multiple LEDs).

By adjusting the temperature of the sensing material 212, the heating element 220 adjusts the sensing characteristics of the sensing material 212. For example, in some cases the heating element 220 may adjust the temperature of the sensing material 212 to increase the sensitivity of sensing material 212 toward a specific analyte and/or to increase the speed by which the sensing material 212 responds to that analyte. Conversely, in other cases, the heating element 220 may adjust the temperature of the sensing material 212 to decrease the sensitivity of sensing material 212 toward a specific analyte and/or to decrease the speed by which the sensing material 212 responds to that analyte.

By adjusting the intensity and/or wavelengths of light incident on the sensing material 212, the lighting element 230 adjusts the sensing characteristics of the sensing material 212. For example, the lighting element 230 may adjust the intensity and/or wavelengths of the light incident of the sensing material 212 to increase the sensitivity of sensing material 212 toward a specific analyte and/or to increase the speed by which the sensing material 212 responds to that analyte. Conversely, in other cases, the lighting element 230 may adjust the intensity and/or wavelengths of the light incident of the sensing material 212 to decrease the sensitivity of sensing material 212 toward a specific analyte and/or to decrease the speed by which the sensing material 212 responds to that analyte.

In some examples, the heating element 220 may be a single heating element such that one or more sensors 200 share a single heating element. Likewise, the lighting element 230 may be a single lighting element such that one or more sensors 200 share a single lighting element. The various sensors 200 comprising the sensor array 100 may contain the same or different sensing elements 210. In this way, in some examples, the sensor array 100 may contain a plurality of different sensing elements 210. For example, the sensor array 100 may include 8, 16, 60, 118, or 128 different sensing elements 210. Likewise, the various sensors 200 comprising the sensor array 100 may each employ different types of heating elements 220. For example, some sensors 200 may employ microheaters made of one material, while other sensors may employ microheaters made of another material. Similarly, the various sensors 200 comprising the sensor array 100 may each employ different types of lighting elements 230. For example, some sensors 200 may employ lighting elements that include LEDs, while other sensors employ lighting elements that include lasers.

As shown in FIG. 1, the controller 300 of the detection device 10 comprises a readout unit 310, a heating control unit 320, a lighting control unit 330, an analysis unit 340, and a communication unit 350. The readout unit 310, heating control unit 320, lighting control unit 330, analysis unit 340, and communication unit 350 each execute computer-executable instructions stored in a memory. The readout unit 310, heating control unit 320, lighting control unit 330, analysis unit 340, and communication unit 350 may comprise multiple separate devices or a single device such as a single microcontroller. Additionally, the controller 300 may be integrated in whole or in part within the detection system 10 or may comprise a separate device that communicates, for example, via a wired or wireless connection, with the detection device. In some examples, the controller 300 is electronically coupled to an external component (e.g., a computer or processor that executes computer-executable instructions stored in a memory).

The readout unit 310 measures the electrical properties of each sensing material 212. For example, the readout unit may measure the resistance, capacitance, and/or impedance of a sensing material 212 by measuring the resistance, capacitance, and/or impedance between its electrode pair 211. The readout unit 310 may make a single measurement or a sequence of measurements so as to record the variation in resistance, capacitance, and/or impedance over time. The readout unit 310 may store the measurements in computer readable media for processing by the analysis unit 340.

The heating control unit 320 controls the temperature of the heating elements 220. The heating control unit 320 provides commands to each individual heating element 220 causing that heating element 220 to maintain the temperature of the corresponding sensing material 212 according to a temperature profile. The temperature profile characterizes the desired variation in the temperature of the sensing material 212 during the period of sensing. The heating control unit 320 contains (e.g., in computer readable media) stored temperature profiles for each different sensing material 212 and for each target analyte. In this way, the heating control unit 320 controls the temperature of each sensing material 212 so as to achieve conditions suited for detecting the target analyte or analytes. These temperature profiles may be generated according to an iterative process that leverages machine learning, as described in greater depth below.

The lighting control unit 330 controls the illumination produced by the lighting elements 230. The lighting control unit 330 provides commands to each individual lighting element 230 causing that lighting element 230 to illuminate the corresponding material 212 according to an illumination profile. The illumination profile characterizes the desired variation in wavelength composition and intensity of light incident on the sensing material 212 during the period of sensing. The lighting control unit 330 contains (e.g., in computer readable media) illumination profiles for each different sensing material 212 and each target analyte. In this way, the lighting control unit 330 controls the incident light on each sensing material 212 so as to achieve conditions suited for detecting the target analyte or analytes. These illumination profiles may be generated according to an iterative process that leverages machine learning, as described in greater depth below.

The analysis unit 340 processes the measurement data from the readout unit 310 to identify the presence or absence of target analytes. In some embodiments, the analysis unit 340 employs a machine learning model that receives as input the measurement data from the readout unit 310 and produces as output an identity and/or concentration of the detected analytes. By considering the electrical responses of multiple sensors 200 comprising the array 100, this machine learning model can achieve higher accuracy at determining the identity and/or concentration of analytes than is typically possible by considering the electrical response of only a single sensor.

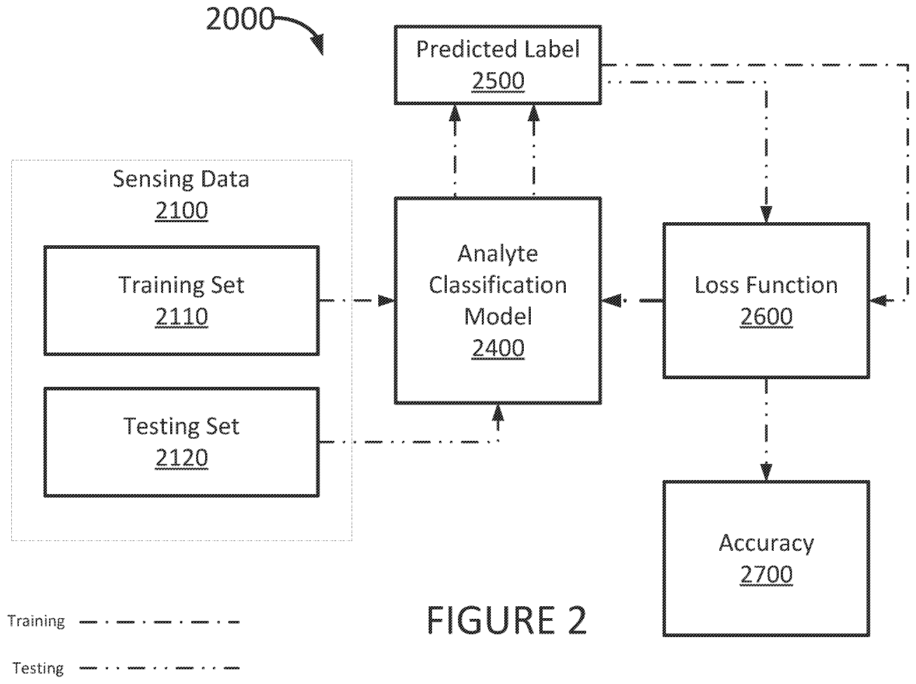

FIG. 2 is a block diagram illustrating an example system 2000 for training a machine learning model to identify analytes and their concentrations. To distinguish such a machine learning model from other machine learning models used herein, a machine learning model trained according to system 2000 is referred to herein as an “analyte classification model.” The method of system 2000 may be performed on a separate computer with the resulting trained model being transferred to the detection device 10. Alternatively, the method of system 2000 may be performed directly on the detection device 10. As shown, the system 2000 trains the machine learning model according to an iterative process in which the model is first trained and then tested for accuracy. The flow of components in the initial training stage is indicated in FIG. 2 by dash-dot-dash lines, and the flow of components in the subsequent testing stage is indicated in FIG. 2 by dash-dot-dot-dash lines.

As shown in FIG. 2, the system 2000 includes sensing data 2100 and analyte classification model 2400. The sensing data 2100 are generated by exposing the sensor array 100 to known analytes (e.g., both individual analytes and combinations of analytes) in known concentrations, and the electrical responses of the sensors 200 are measured by the readout unit 310 and recorded. Each such exposure comprises a sensing example, such that the sensing data 2100 include multiple sensing examples. In addition to the electrical response data from the readout unit, each sensing example also contains data indicating the temperature and illumination profiles used for each sensor 200. Each sensing example is labeled with the known analytes to which the array was exposed (both the identity of the analytes and their concentrations). A plurality of such sensing examples is generated for various temperature profiles, illumination profiles, and analytes and concentrations.

The sensing data 2100 is divided into a training set 2110 and a testing set 2120. The training set 2110 may include a pre-determined portion of the sensing examples from the sensing data 2100 (e.g., 70% of the examples, etc.), and is used to train the analyte classification model 2400. The training process tunes the model 2400, for example, by adjusting the parameters of the model, to enable it to accurately predict the labels of the examples (e.g., the analytes present and their concentrations). During training, the model 2400 “learns” which labels correspond to a particular array response (e.g., the change in electrical properties of each sensor 200 as measured by readout unit 310) for a particular operating condition (e.g., the temperature profile and illumination profile of each sensor 200). The testing set 2120 may include the portion of the sensing data 2100 not used in the training set. The testing set 2120 is used to evaluate the accuracy of the machine learning model. Because the testing set 2120 is used for testing the model (e.g., to determine how accurate the model is), this data is not used for training the model.

The analyte classification model 2400 is characterized by its parameters (e.g., the weights of an artificial neural network, the tests of a decision tree, the coefficients of a regression model, etc.). In some implementations, an iterative process is used to identify optimal (e.g., most-aligned with a goal of the model 2400) values for the parameters of the model 2400. During an iteration of training, the model 2400 computes a predicted label 2500 for each sensing example from the training set 2110. This predicted label 2500 for a particular sensing example may be a predicted identity of the analytes present and their concentrations. In one implementation, the predicted label 2500 of the model 2400 may have the form of a one-dimensional vector in which a value of zero indicates the absence of a target analyte, whereas a non-zero value indicates the concentration of an analyte that has been detected.

The predicted label 2500 is input into a loss function 2600, which compares the predicted label 2500 to the corresponding “true” label (e.g., the label originally associated with the respective entry from the training set on which the model 2400 based its predicted label). For example, if the predicted label 2500 for the analyte is ammonia at 10 parts per million (ppm), while the true label from the training set 2110 is ammonia at 8 ppm, there is an error of 2 ppm. This comparison may be repeated for any number of sensing examples in the training set 2110. For example, the comparison may be made for all sensing examples in the training set 2110, or for a pre-determined number (e.g., 10). The loss function 2600 then computes a value based on the errors for each compared sensing example. To complete the training iteration, the value computed by the loss function 2600 is leveraged to adjust the parameters of the model 2400 to reduce the computed value of the loss function 2600, thereby reducing the aggregate error of the model 2400. Multiple such iterations are performed to minimize the computed value of the loss function 2600, thereby minimizing the aggregate error of the model 2400. In this way, the loss function 2600 is used with the training data 2110 to improve the prediction accuracy of the model 2400.

Once the model has been trained, the testing data is used to compute an accuracy 2700 of the model. The trained model 2400 is used to predict the label for each testing example. The predicted label of each testing example is compared to the corresponding “true” label from the testing set and any difference is represented as an error value. Loss function 2600 computes a value that combines the error values for all of the testing examples. Although loss function 2600 is shown as used in this example, other loss functions can be used. The value of the loss function 2600 on the testing data defines the accuracy 2700 of the model, which for convenience, may be referred to as the “analyte classification accuracy”.

The trained model 2400 is embedded in the analysis unit 340 to identify analytes and their concentrations.

While reference may be made to the analyte classification model 2400 comprising a single machine learning model, it is understood that there may be multiple machine learning models. For example, in one implementation a separate machine learning model may be made for each individual sensor 200. In this example, an additional machine learning model then aggregates the outputs of those individual models to determine the analytes present and their concentrations. Likewise, in some implementations there may be a separate machine learning model for each choice of a temperature and illumination profile. In other implementations, one machine learning model may be used for multiple temperature and illumination profiles. In some implementations, one machine learning model (or models) may identify which analytes are present and which are absent, while another model (or models) may determine the concentration of those analytes that were identified as present.

While the process of training a machine learning model is described in terms of learning model parameters, it is understood that the machine learning includes the use of both parametric machine learning algorithms (e.g., artificial neural networks, regression, etc.) and non-parametric learning algorithms (e.g., k-nearest neighbor, decision trees, etc.). Thus, training a model is to be understood generally as identifying the quantities, such as the weights of an artificial neutral network or the tests of a decision tree, that define the model. Such training may or may not include iteration.

As shown in FIG. 2, the sensing data 2100 are divided into a training set 2110 and a testing set 2120. It is understood, however, that in some examples the sensing data 2100 may be divided into three sets: a training set, a testing set, and a validation set. The training and testing sets in these examples are analogous to training set 2110 and testing set 2120 respectively. The validation set is used for an additional level of training. In this additional level, the validation data set is used, for example, to compare the performance of alternative machine learning algorithms (e.g., artificial neural networks, decision trees, etc.) or to tune hyperparameters such as the learning rate or the number of nodes for an artificial neural network. The performance of a model on the validation set is computed using a loss function (e.g., loss function 2600) just as with the training and testing sets.

The machine learning models may operate directly on the measurement data from the readout unit 310, e.g., raw data. Alternatively, the data may be preprocessed. For example, preprocessing may include considering the difference between that data and sensing data obtained in the absence of the analytes (e.g., data obtained from measurements in clean, dry air or pure nitrogen). As each set of sensing data comprises a time series of electrical measurements, this comparison is computed for every pair of corresponding measurements. For example, the analysis unit 340 may compute the ratio of, or the difference between, corresponding measurements. Likewise, features may be computed from the measurement data. Features may include, for example, the time rate of change (slope) of the electrical response, the frequency content of the response (e.g., coefficients of a Fourier transform), the time to reach the peak response, the magnitude of the peak response, etc. The features may be computed either from the raw data or the preprocessed data. In this way, the machine learning models may operate on raw data, preprocessed data, computed features, or a combination of those. Data preprocessing and feature computation are performed by the analysis unit 340. This analysis unit includes computer-executable instructions stored in a memory. The analysis unit also includes model parameters for the machine learning models and other data which is stored in computer readable media.

The communication unit 350 provides a user interface for the user of the detection device 10 to operate it, including specifying the target analytes to be detected. The communication unit 350 may also display the results of the detection such as the presence and absence of target analytes and the concentrations of the analytes that are present. The communication unit 350 may also communicate with other devices such as mobile phones, desktop computers, cloud computers, etc. so as to enable the detection device 10 to be operated remotely and/or to transmit detection results.

In some examples, the detection device 10 may be fabricated, in whole or in part, on a printed circuit board (PCB) or flexible polyimide substrate. The sensor array 100 may contain one or more individually addressable sensors 200. In some examples, these sensors are electrical gas sensors. In some examples, a sensor comprises a Micro-Electro-Mechanical System (MEMS). Each electrical gas sensor may include a sensing element 210, a heating element 220, and a lighting element 230. The detection device 10 may include a controller 300 such that the controller 300 is configured to adjust the conditions (e.g., temperature, brightness of light, wavelength spectrum of light, etc.) for the sensing material 212 of each sensing element 210. The sensing material 212 may comprise a semiconducting sensing material that electrically bridges an electrode pair 211. In some examples, the electrode pair 211 is a pair of source-drain electrodes. In some examples, the controller 300 may be fabricated on a PCB while the sensor array 100 is fabricated on a flexible polyimide substrate.

In some examples, the heating of the sensing material 212 by heating element 220 and/or the illumination of the sensing material 212 by the lighting element 230 may facilitate detection of the target analyte by inducing oxidation and/or reduction of that analyte. In some examples, this heating and/or illumination of the sensing material 212 may facilitate detection of the target analyte by inducing chemical and/or physical changes in the absorption and/or desorption properties of the sensing material 212. In some examples, this heating and/or illumination of the sensing material 212 may facilitate detection of the target analyte by altering the baseline electrical properties (e.g., the Fermi level, grain boundary potential barrier, work function, dielectric constant, etc.) of the sensing material 212. In some examples, this heating and/or illumination of the sensing material 212 may facilitate detection of the target analyte by altering the surface reactivity of the sensing material 212. In some examples, the illumination of the sensing material 212 by lighting element 230 may alter the amount of photogenerated free electron-hole pairs in the sensing material 212, thus facilitating the detection of the target analyte.

In some implementations, the temperature profiles used by the heating control unit 320 and the illumination profiles used by the lighting control unit 330 are determined from a training process (the “profile training process”). This process identifies temperature profiles and illumination profiles to maximize the sensing performance of the sensor array 100 for detecting particular analytes. During a step of the training process, a candidate temperature profile and candidate lighting profile are generated for each sensor 200. In some implementations, these candidate profiles may be generated by selecting them from a set of standard profiles such as ramp profiles, square wave profiles, sinusoidal profiles, step profiles, or combinations of these, for example. Next, the sensor array 100 is exposed to analytes in various combinations and concentrations. The response data from the sensor array 100, as measured by the readout unit 310, is used to train an analyte classification model using the process 2000 of FIG. 2. The analyte classification accuracy of the trained model is then computed. Multiple such training steps are performed and the temperature and illumination profiles which achieve the highest analyte classification accuracy for a particular analyte or analytes are identified. These profiles are then stored in the heating controller 320 and lighting controller 330 for use when the detection system is employed to detect that analyte or combination of analytes.

In some implementations, the profile training process is combined with a machine learning process that produces a profile performance model. The profile performance model is a machine learning model that relates the parameters of the temperature and illumination profiles to the analyte classification accuracy that these profiles achieve. More specifically, each step of the profile training process produces a training example for training the profile performance model. A training example comprises a set of parameter values that define the temperature and illumination profiles such as their shape (e.g., square wave or sinusoid when plotted on a 2-dimensional graph with time as the x-axis), amplitude, wavelength content, rate of change, etc. The training example is also labeled by the analyte classification accuracy achieved in that step of the profile training process. The training examples are used to train a machine learning model that takes as input temperature and illumination profile parameters and produces as output the predicted analyte classification accuracy. This model is then used to efficiently identify temperature and illumination profiles (e.g., temperature and illumination profile parameters) to maximize the analyte classification accuracy. In some implementations, the temperature and illumination profile parameters identified in this fashion are used to produce new profile training steps which are then used as new training examples for the profile performance model so as to create an improved model. The improved model is then used to identify new temperature and illumination profiles, further maximizing the analyte classification accuracy. In this fashion, the performance of the profile performance model and the performance of the temperature and illumination profiles found may be iteratively improved.

In some implementations, a machine learning model is used to identify optimal (e.g., most sensitive, most reactive, etc.) sensing materials 212 for detecting particular analytes. The properties of the sensing materials directly affect the sensing performance of those materials. Machine learning is used to create a model, called the material performance model, that relates a material's properties and/or synthesis parameters to its sensing performance. The material properties may include characteristics of the material such as the morphology (e.g., diameter, length), composition (e.g., dopant concentration), structure (e.g., crystal substructure, crystallinity, grain size, preferred crystal orientation), electrical properties (e.g., band gap, carrier concentration, carrier type, carrier mobility), optical properties (e.g., band-gap, color), chemical and physical properties (e.g., surface area, adsorption/desorption kinetics), etc. The synthesis parameters characterize the synthesis process used to fabricate the sensing material and include parameters of the electrospinning process (e.g., voltage, flow rate, temperature, etc.), parameters of the thermal treatment (e.g., temperature and processing time), and parameters of any other fabrication processes used.

To train the material performance model multiple versions of a material are fabricated such that they differ in one or more material properties and/or synthesis parameters. A sensor 200 is then fabricated from each version of the sensing material and each such sensor is then integrated into an array 100. In some examples, the array 100 is unique to the sensor 200. In other examples, the array 100 is a previously-generated array 100 re-used for a new iteration of training the material performance model. An analyte classification model is created for each such array 100 (e.g., according to system 2000 of FIG. 2). Each array corresponds to a training example for the material performance model. A training example is characterized by the material properties and/or synthesis parameters of the material variant contained in that array. The training example is labeled with the analyte classification accuracy achieved by the array. The training examples are used to train a machine learning model that takes as input the material properties and/or synthesis parameters and produces as output the predicted analyte classification accuracy. This model is then used to efficiently identify material variants (e.g., material properties and/or synthesis parameters) to maximize the analyte classification accuracy. In some implementations, the material properties and/or synthesis parameters identified in this fashion are used to produce new sensing material variants which are then used as new training examples for the material performance model so as to create an improved model. The improved model is then used to identify new material properties and/or synthesis parameters to further maximizing the analyte classification accuracy. In this fashion, the performance of the material performance model and the sensing performance of the sensing materials may be iteratively improved.

For example, to identify the relationship between thermal treatment parameters and the analyte classification accuracy, multiple sensing materials are constructed differing in the values of the thermal treatment parameters. Sensors 200 are created from these sensing materials and installed on sensor arrays 100. Each array is subjected to known analytes in known concentrations such that an analyte classification model is produced. In this way each array corresponds to a training example which contains the values of the thermal treatment parameters used for that material variant and which is labeled with the analyte classification accuracy achieved by that variant. The examples are used to train a material performance model that takes as input values of the thermal processing parameters and produces as output a predicted analyte classification accuracy. This model is then used to identify thermal processing parameters to maximize the analyte classification accuracy and thus maximize the sensing performance.

The profile performance model and material performance model are trained using a process similar to the process 2000 in FIG. 2 used to train the analyte classification model 2400, such that the profile performance model or material performance model may be substituted for the analyte classification model 2400 in FIG. 2. For example, for each of the profile performance model or material performance model, the data (e.g., sensing data 2100) are divided into training (e.g., training set 2110) and testing (e.g., testing set 2120) data, predicted values are produced (e.g., predicted label 2500), and a loss function (e.g., loss function 2600) is used. Also, it is understood that the profile performance model and material performance model can be trained sequentially or simultaneously so that temperature and illumination profiles and sensing materials can be optimized sequentially or simultaneously.

While reference is made to training machine learning models to maximize analyte classification accuracy, it is understood that the machine learning models could also be used to maximizer or minimize other performance measures such as speed of response, lower limit of detection, response time, recovery time, etc. In these alternative embodiments, the sensing examples are instead labeled with the desired performance measure, and the loss function (e.g., loss function 2600 of FIG. 2) considers the error between the predicted value of that performance measure and the true value of that measure.

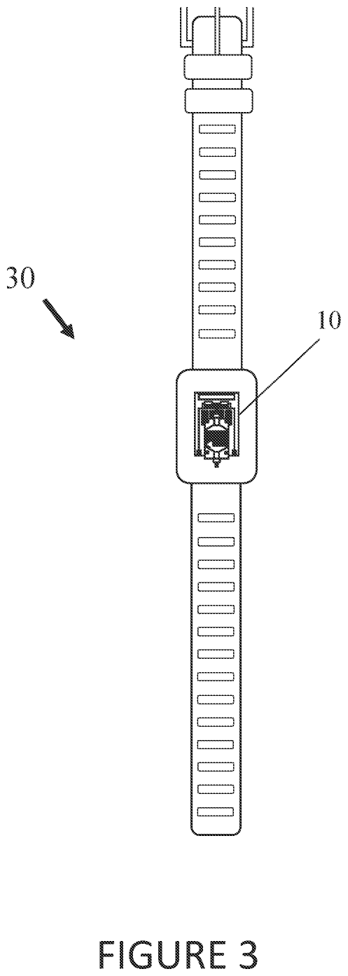

FIG. 3 is a diagram of an example wearable device 30 that includes the detection device 10 of FIG. 1. As shown in FIG. 3, the detection device 10 may be incorporated into a portable or wearable device (e.g., a watch). The wearable device 30 may be electronically coupled to an external device (e.g., a computer, mobile phone, or other electronic device that includes a processor which executes computer-executable instructions stored in a memory). The external device may be configured to interact with a user such that the user uses the external device to select an analyte for the detection device 10 to detect and the external device then displays the results (detected analytes and concentrations) from the device 10. In some examples, the wearable device 30 may include a microprocessor and a graphical user interface such that the user may interact directly with the wearable device 30.

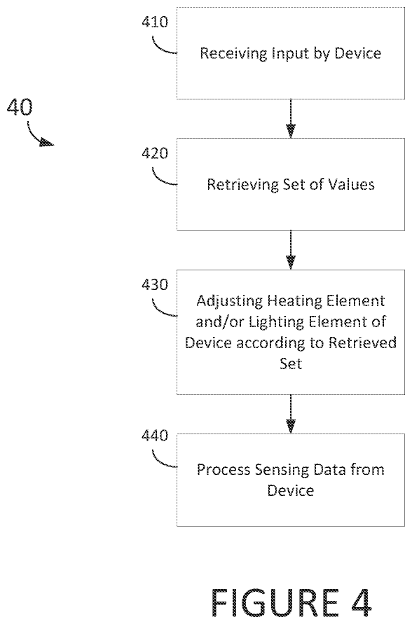

FIG. 4 is a is a flow chart illustrating an example method 40 of detecting analytes. The method 40 may be performed, in whole or in part, by the detection device 10 and, in particular, the sensor 200.

In step 410, an input of a user-selected parameter is received by a user input element coupled to the detection device 10. In some examples, the user-selected parameter is the selection of a target analyte to be detected, such as ammonia, nitrogen dioxide, nitric oxide contained in air, nitric oxide contained in nitrogen, carbon monoxide, nitrous oxide, methyl nonafluorobutyl ether in trans-1,2-dichloroethylene, methoxy-nonafluorobutane, 1,1,2-Trichloro-1,2,2-trifluoroethane, acetone, ethanol, toluene, ethylbenzene, xylene, benzene, or methane. In some examples, the user input element is included on a graphical user interface (GUI). The GUI may be on a portable electronic device with a touchscreen display. In some examples, the detection device 10 may be incorporated into the portable electronic device, as shown in FIG. 3

In step 420, a set of values from a dataset stored in computer readable media is retrieved. In some examples, the set of values comprises temperature profiles for the heating elements 220 and illumination profiles for the lighting elements 230. These target sensing conditions may define the most efficient, fastest, and/or most sensitive conditions for detecting the target analyte.

In step 430, the heating elements 220 are controlled by the heating control unit 320 according to the retrieved temperature profiles and the lighting elements 230 are controlled by the lighting control unit 330 according to the retrieved illumination profiles. Simultaneously, the readout unit 310 measures the electrical responses of the sensing elements 210. In some examples, the heating control unit 320 receives a set of values from the dataset and, in response, the heating control unit 320 may issue commands to the heating elements 220 of the one or more sensors 200. Likewise, in some examples the lighting control unit 330 receives a set of values from the dataset and, in response, the lighting control unit 330 may issue commands to the lighting elements 230 of the one or more sensors 200. In response to receiving commands from the heating control unit 320, a heating element 220 may increase electrical flow through the microheater (or other suitable heating element) such that the metals or other materials that comprise the microheater emit heat thereby increasing the temperature provided by the heating element 220. In other cases, in response to receiving commands from heating control unit 320, the heating element 220 may decrease electrical flow through the microheater (or other suitable heating element) such that the metals or other materials that comprise the microheater emit less heat thereby decreasing the temperature provided by the heating element 220. In response to receiving commands from the lighting control unit 330, a lighting element 230 may increase electrical flow through the LEDs (or other suitable light source) such that the electrical current illuminates the LEDs, thereby increasing the brightness provided by the lighting element 230. In other cases, in response to receiving commands from the lighting control unit 330, a lighting element 230 may decrease electrical flow through the LEDs (or other suitable light source), thereby decreasing the brightness provided by the lighting element 230.

In step 440, the analysis unit 340 processes the sensing data from the readout unit 310. The analysis unit 340 then retrieves a machine learning model and uses it to process the data and identify the analytes present and their concentrations. The machine learning model may operate directly on the sensing data from the readout unit 310 or the analysis unit 340 may first pre-process the data and/or compute features from that data. Data from a single sensor 200 or a collection of sensors may be indicative of a particular target analyte. The presence and absence of analytes, and the concentration of any detected analytes, may be displayed, for example on the GUI. The GUI may be on a portable electronic device with a touchscreen display, such as device shown in FIG. 2.

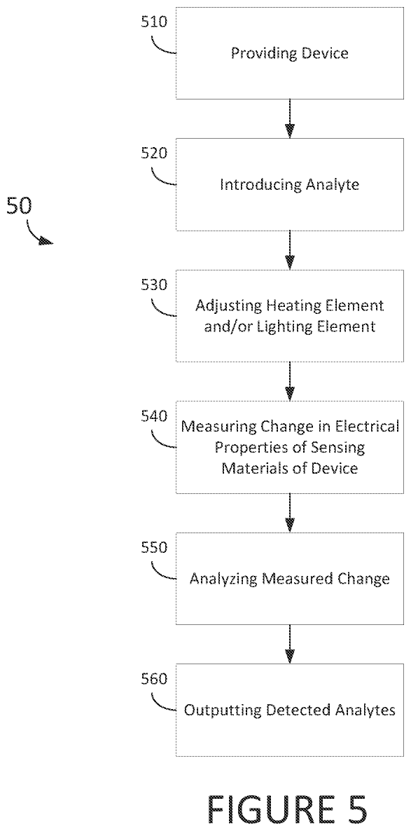

FIG. 5 is a flow chart illustrating an example method employing a machine learning model for analyte classification. The method 50 may be performed, in whole or in part, by the detection device 10.

In step 510, the detection device 10 is provided. The detection device may be the detection device 10 of FIG. 1.

In step 520, the detection device 10 is introduced to an analyte. The analyte may be any a substance whose chemical constituents are being identified and measured. The substance may be a chemical substance (e.g., ammonia hydrogen sulfide, etc.), an odor, or smell, for example, such the analyte may be any sensible gaseous molecules.

In step 530, at least one of the heating element 220 or the lighting element 230 of a sensor 200 is adjusted. A heating element 220 is adjusted according to a temperature profile which may be indicative of a temperature of the heating element 220. A lighting element 230 is adjusted according to an illumination profile which may be indicative of a brightness or wavelength composition of the light emitted by the lighting element 230.

In step 540, changes in electrical properties are measured. The nearby chemical environment causes changes in the electrical properties of the sensing materials 212. In this step, those changes in electrical properties are measured for at least one sensing material 212. In the present example, the readout unit 310 measures the change in electrical properties of the sensing material 212 of each of the plurality of sensors 200 comprising the sensor array 100.

In step 550, the analysis unit 340 analyzes the sensing data obtained in step 540. The analysis unit 340 inputs the data into a trained machine learning model (e.g., the analyte classification model 2400 of FIG. 2). The analysis unit may preprocess the data before inputting it into the machine learning model, for example, by computing the ratio of each measurement to a corresponding measurement made in a reference atmosphere such as clean, dry air or pure nitrogen. Additionally, the analysis unit may compute features from the data and input these to the machine learning model instead of, or in combination with, the data. The features might include, for example, the time rate of change of the electrical response, the frequency content of the response (e.g., coefficients of a Fourier transform), the time to reach the peak response, and the magnitude of the peak response. The machine learning model can employ any suitable machine learning algorithm including, for example, artificial neural networks, decision trees, k-nearest neighbors, look up tables, etc.

In step 560, the machine learning model of step 550 produces as output a list of the analytes detected along with their concentrations.

Some portions of the detailed descriptions of this disclosure have been presented in terms of procedures, logic blocks, processing, and other symbolic representations of operations on data bits within a computer or digital system memory. These disclosures and representations are the means used by those of ordinary skill in the art of data processing to most effectively convey the substance of their work to others of ordinary skill in the art. A procedure, logic block, process, etc., is herein, and generally, conceived to be a self-consistent sequence of steps or instructions leading to a desired result. The steps are those requiring physical manipulations of physical quantities. Usually, though not necessarily, these physical manipulations take the form of electrical or magnetic data capable of being stored, transferred, combined, compared, and otherwise manipulated in a computer system or similar electronic computing device. For reasons of convenience, and with reference to common usage, such data is referred to as bits, values, elements, symbols, characters, terms, numbers, or the like, with reference to various presently disclosed examples. It should be borne in mind, however, that these terms are to be interpreted as referencing physical manipulations and quantities and are merely convenient labels that should be interpreted further in view of terms commonly used in the art. Unless specifically stated otherwise, as apparent from the discussion herein, it is understood that throughout discussions of the present example, discussions utilizing terms such as “determining” or “outputting” or “transmitting” or “recording” or “locating” or “storing” or “displaying” or “receiving” or “recognizing” or “utilizing” or “generating” or “providing” or “accessing” or “checking” or “notifying” or “delivering” or the like, refer to the action and processes of a computer system, or similar electronic computing device, that manipulates and transforms data.

While this disclosure has described certain examples, it will be understood that the claims are not intended to be limited to these examples except as explicitly recited in the claims. On the contrary, the instant disclosure is intended to cover alternatives, modifications and equivalents, which may be included within the spirit and scope of the disclosure. Furthermore, in the detailed description of the present disclosure, numerous specific details are set forth in order to provide a thorough understanding of the disclosed examples. However, it will be obvious to one of ordinary skill in the art that systems and methods consistent with this disclosure may be practiced without these specific details. In other instances, well known methods, procedures, components, and circuits have not been described in detail as not to unnecessarily obscure various aspects of the present disclosure.

1. A detection device to detect analytes comprising:
a sensor array comprising a plurality of sensors, the plurality of sensors comprising:
a sensing element;
a heating element; and
a lighting element; and
a controller, wherein the controller is communicatively coupled to the sensor array, the heating element, and the lighting element, and configured to adjust at least one of the heating element or the lighting element based on a temperature profile of the heating element and an illumination profile of the lighting element.

2. The detection device of claim 1, wherein the sensing element comprises:
an electrode pair;
a sensing material; and
a permselective membrane.

3. The detection device of claim 1, wherein the sensing element comprises tungsten trioxide-based nanofibers.

4. The detection device of claim 3, wherein the tungsten trioxide-based nanofibers are doped with a noble metal.

5. The detection device of claim 1, wherein the sensing element comprises tin dioxide-based nanofibers.

6. The detection device of claim 5, wherein the tin dioxide-based nanofibers are doped with a noble metal.

7. The detection device of claim 1, wherein the heating element comprises a plurality of heating elements and the lighting element comprises a plurality of lighting elements, such that each of the plurality of sensors comprises a respective heating element of the plurality of heating elements and a respective lighting element of the plurality of lighting elements.

8. The detection device of claim 2, wherein the sensing material comprises a material formed via an electrospinning process.

9. The detection device of claim 8, wherein the electrospinning process comprises:
a solution;
a syringe for holding and dispensing the solution; and
a collector for collecting the solution dispensed from the syringe, wherein:
the solution is polymer based,
the syringe is under a high-voltage electric field,
the collector is electrically grounded, and
the solution is ejected by the syringe toward the collector, such that the solution solidifies into nanofibers.

10. The detection device of claim 1, wherein:
the temperature profile and the illumination profile are determined by a machine learning model trained to identify a temperature value and an illumination value that correspond to a sensitivity value for the sensing element.

11. The detection device of claim 10, wherein the sensitivity value for a respective sensing element is based on at least one of a quantity, a timing, or a length of a detected change in an electrical property of the sensing element.

12. The detection device of claim 1, wherein the sensing element comprises tungsten trioxide-based nanofibers.

13. The detection device of claim 1, wherein the sensing element comprises tin dioxide-based nanofibers.

14. A method of detecting analytes, comprising:
receiving an input of a user-selected parameter, wherein the input is received by a user input element coupled to a device, the device comprising:
a sensor array comprising a plurality of sensors, the plurality of sensors comprising:
a sensing element;
a heating element; and
a lighting element; and
a controller, wherein the controller is communicatively coupled to the sensor array, the heating element, and the lighting element;
retrieving a set of values from a data set stored in computer readable media, wherein the set of values is related to the user-selected parameter;
adjusting at least one of the heating element and the lighting element based on the retrieved set of values; and
generating, by the controller, a response, wherein the response is received by the user input element.

15. The method of claim 14, wherein the user-selected parameter is a target analyte.

16. The method of claim 14, wherein the set of values comprises a temperature profile and an illumination profile.

17. The method of claim 16, wherein:
the temperature profile and the illumination profile are determined by a machine learning model trained to identify a temperature value and an illumination value that correspond to a sensitivity value for the sensing element.

18. The method of claim 17, wherein the sensitivity value for a respective sensing element is based on at least one of a quantity, a timing, or a length of a detected change in a electrical property of the sensing element.

19. A method for analyte classification, the method comprising:
providing a device comprising:
a sensor array comprising a plurality of sensors, the plurality of sensors comprising:
a sensing element;
a heating element; and
a lighting element; and
a controller communicatively coupled to the sensor array, the heating element, and the lighting element, and configured to adjust at least one of the heating element or the lighting element based on a temperature profile of the heating element and an illumination profile of the lighting element;
receiving, by the controller, an indication of a target analyte;
retrieving, by the controller, the temperature profile and the illumination profile associated with the target analyte;
introducing the device to an unknown target substance; and
determining, via a trained machine learning model stored in the controller, a concentration of the target analyte in the unknown target substance.

20. The method of claim 19, wherein the temperature profile and the illumination profile are determined by a second machine learning model trained by:
introducing a known analyte of a plurality of known analytes to a known sensing element of a plurality of known sensing elements at a known temperature of a plurality of known temperatures and with a known illumination of a plurality of known illuminations;
recording a change in electrical property of the known sensing element in response to the known analyte;
repeating the introduction and recording steps with at least one different known parameter from the plurality of known analytes, the plurality of known sensing elements, the plurality of known temperatures, or the plurality of known illuminations to generate a table of known profiles; and
training the machine learning model based on the table of known profiles.

21. A method for training a machine learning model comprising:
generating a plurality of materials, each of which have at least one different parameter from the other of the plurality of materials;
providing a plurality of sensor arrays, each comprising:
a sensing element comprising at least one of the plurality of generated materials;
a heating element; and
a lighting element;
exposing each of the plurality of sensor arrays to an analyte and monitoring the responses of the plurality of sensor arrays; and
generating a training dataset for the machine learning model by associating the responses of the plurality of sensor arrays with a respective at least one of the plurality of generated materials.

22. The method of claim 21, wherein the trained machine learning model is configured to provide an estimated sensing performance parameter in response to receiving a proposed sensing material.

23. The method of claim 22, wherein the estimated sensing performance parameter comprises at least one of:
a speed of response by the proposed sensing material to an exposure,
lower limit of detection of the proposed sensing material to the exposure,
response time by the proposed sensing material, or
recovery time between exposures by the proposed sensing material.
